(19) United States
(12) Patent Application Publication
(10) Pub. No.: US 2026/0080994 A1
(43) Pub. Date: Mar. 19, 2026

(54) SelectMed

(71) Applicants: Patricia Hill, Moncks Corner, SC (US); Morgan Hill, Moncks Corner, SC (US); Luke Hill, Moncks Corner, SC (US); Jack Hill, Moncks Corner, SC (US)

(72) Inventors: Patricia Hill, Moncks Corner, SC (US); Morgan Hill, Moncks Corner, SC (US); Luke Hill, Moncks Corner, SC (US); Jack Hill, Moncks Corner, SC (US)

(21) Appl. No.: 18/884,741

(22) Filed: Sep. 13, 2024

(51) Int. Cl. G16H 20/10; G16H 80/00

(52) U.S. Cl. CPC G16H 20/10; G16H 80/00

(57) ABSTRACT

SelectMed will streamline communication between patients, doctors, and pharmacists. SelectMed provides patients with a secure, personalized portal, where their diagnosis and treatment options are integrated. Doctors, patients and pharmacists can communicate, upload results and provide follow up. Once a doctor enters the patient's diagnosis, SelectMed generates approved medications for that condition. Doctors then select and rank multiple medications. There is also an option for doctors to add medications not on the list.

The patient and pharmacist then choose the medication covered by insurance and within their budget. Currently doctors write prescriptions without knowing the patient's insurance coverage or costs. Patients then face delays due to insurance issues, unavailability, or cost. This leads to additional doctor visits/phone calls, delaying treatment.

SelectMed enables doctors to pre-approve a range of medications, giving pharmacists the flexibility to dispense the best option based on coverage and affordability, saving time, reducing costs, and improving health faster.

### SELECTMED PORTAL BACKGROUND

I will develop a computer program, SelectMed, that allows the patient, doctor and pharmacist a platform to communicate. Each patient will have a personalized portal within SelectMed. When the doctor enters the patient's diagnosis in their portal, all the medications that treat that diagnosis will populate along with an option for the doctor to write in additional medications. The doctor will then rank/select the medications that they approve for the patient to have filled by the pharmacist. When the patient gets to the pharmacy, the patient and pharmacist will work together to find which medication that is covered by their insurance and in their budget. This will save the patient money and time. Currently, the doctor can only write one medication on the prescription pad. Doctors don't know which insurance companies cover which medications or the cost of those medications. Currently, the doctor gives the patient a prescription written on a piece of paper, the patient has to drive to the pharmacy and drop the prescription off. Typically, hours later the pharmacist notifies the patient that their insurance company does not cover the prescription, the pharmacy doesn't have the medication or the cost is very high. The patient then has to call the doctors office to get a new prescription written, and then called in to the pharmacy or make another appointment to discuss other medications with the doctor, which takes more time and money. When the doctor has the ability to rank/select the medication on the patient's portal, SelectMed, that they approve of, then the pharmacist can disperse the medication to the patient based on their insurance coverage and budget. This will save time and money for the patient, doctor and pharmacist and allow the patient to get their medication more quickly.

The SelectMed portal is a comprehensive solution designed to streamline communication between patients, doctors, and pharmacists. This innovative platform provides each patient with a personalized portal, where their medical diagnosis and treatment options are seamlessly integrated. There is also a section on the portal where the patient, doctor and pharmacist can communicate with each other, upload test results and provide follow up information.

Once a doctor enters the patient's diagnosis into the system, SelectMed automatically generates a list of approved medications for that condition. Doctors can then select and rank multiple medications that are appropriate for the patient, including options to manually add medications if needed.

1. At the pharmacy, the patient and pharmacist collaborate to choose the medication that is covered by the patient's insurance and fits within their budget. This process reduces inefficiencies, helping patients avoid unexpected costs and delays in obtaining their medications.

2. Currently, the prescription process is fragmented: doctors write a single medication on a prescription pad without knowing the patient's insurance coverage or medication costs. Patients must drive to the pharmacy, drop off their prescription, and often face long delays due to insurance issues, medication unavailability, or cost concerns. This can lead to additional doctor visits or phone calls to revise the prescription, further delaying the patient's treatment.
SelectMed solves these issues by enabling doctors to pre-approve a range of medications within the patient's portal, giving pharmacists the flexibility to dispense the most suitable option based on coverage and affordability. This system saves time, reduces costs, and ensures that patients receive their medications more efficiently, benefiting all parties involved.
